(19) United States
(12) Patent Application Publication
(10) Pub. No.: US 2022/0106402 A1
(43) Pub. Date: Apr. 7, 2022

(54) ANTIBODY

(71) Applicant: KING'S COLLEGE LONDON, London (GB)

(72) Inventors: Heather J. BAX, London (GB); James F. SPICER, London (GB); Debra H. JOSEPHS, London (GB); Giulia PELLIZZARI, London (GB); Sophia N. KARAGIANNIS, London (GB)

(21) Appl. No.: 17/423,602

(22) PCT Filed: Jan. 17, 2020
(86) PCT No.: PCT/EP2020/051121

§ 371 Date: Jul. 16, 2021

(30) Foreign Application Priority Data
Jan. 18, 2019 (GB) 1900724.4

(51) Int. Cl. C07K 16/30; C07K 16/44; A61P 35/00

(52) U.S. Cl. CPC C07K 16/3046; C07K 16/44; C07K 2317/70; C07K 2317/52; A61P 35/00

(57) ABSTRACT

In one aspect, the present invention relates to an immunoglobulin E (IgE) for use in repolarizing macrophages from a first phenotype to an anti-tumor phenotype in the treatment of cancer in a subject; wherein the first phenotype comprises a quiescent (M0) macrophage phenotype or an anti-inflammatory (M2a) macrophage phenotype; and the anti-tumor phenotype comprises a newly polarized macrophage phenotype characterized by expression of the following cytokines and chemokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and/or interleukin-10 (IL-10).

### FIELD OF THE INVENTION

The present invention relates to the field of cancer therapy, and in particular to antibodies for use in treating cancer in a subject. More particularly the invention relates to antibodies and methods for use in treating cancer by repolarizing macrophages to an anti-tumor phenotype.

### BACKGROUND

Therapeutic antibodies now complement conventional treatments for a number of malignant diseases, but almost all agents currently developed rely on only one of the five major human antibody classes, namely IgG, the most abundant antibody class in the blood (Weiner L M et al (2010) Monoclonal antibodies: versatile platforms for cancer immunotherapy. Nat Rev Immunol 10: 317-327). The human immune system naturally deploys nine antibody classes and subclasses (IgM, IgD, IgG1-4, IgA1, IgA2 and IgE) to perform immune surveillance and to mediate destruction of pathogens in different anatomical compartments. Yet only IgG (most often IgG1) has been applied in immunotherapy of cancers.

One reason may be that IgG antibodies (particularly IgG1), constitute the largest fraction of circulating antibodies in human blood. The choice of antibody class is also based on pioneering work in the late 1980s, comparing a panel of chimaeric antibodies of the same specificity, each with Fc regions belonging to one of the nine antibody classes and subclasses (Bruggemann M et al (1987) Comparison of the effector functions of human immunoglobulins using a matched set of chimeric antibodies. J Exp Med 166: 1351-1361). Antibodies were evaluated for their ability to bind complement and their potency to mediate hemolysis and cytotoxicity of antigen-expressing target cells in the presence of complement. IgG1 was the most effective IgG subclass in complement-dependent cell killing in vitro, while the IgA and IgE antibodies were completely inert.

Subsequent clinical trials with antibodies recognizing the B cell marker CD20 supported the inference that IgG1 would be the subclass best suited for immunotherapy of patients with B cell malignancies such as non-Hodgkin's lymphoma (Alduaij W, Illidge T M (2011) The future of anti-CD20 monoclonal antibodies: are we making progress? Blood 117: 2993-3001). Since those studies, comparisons of anti-tumor effects by different antibody classes have been confined to IgG and IgM in both murine models and patients with lymphoid malignancies, while IgA has been shown to mediate ADCC in vitro and in vivo in mouse models of lymphoma (Dechant M, Valerius T (2001) IgA antibodies for cancer therapy. Crit Rev Oncol Hematol 39: 69-77).

Complement-mediated tumor cell death is now known to be only one of several mechanisms by which antibodies may mediate tumor growth restriction (Weiner G J (2007) Monoclonal antibody mechanisms of action in cancer. Immunol Res 39: 271-278). Known mechanisms include engaging immune effector molecules through their Fc regions to induce immune cell mediated destruction of targeted cells by antibody-dependent cell-mediated cytotoxicity (ADCC) and phagocytosis (ADCP). Antibodies can also act directly on tumor cells to inhibit growth signaling pathways, induce apoptosis, restrict proliferation and cell differentiation of tumor cells, or block tumor cell adhesion and migration. Some antibodies are developed to recognize targets associated with tumor-associated vasculature in order to starve tumors of vital nutrients delivered through blood supply, while others attack immune regulatory targets (e.g. CTLA-4 and PD-1) to enhance T cell activation and overcome immunosuppressive elements of the tumor microenvironment (Ascierto P A et al (2010) Clinical experiences with anti-CD137 and anti-PD1 therapeutic antibodies. Semin Oncol 37: 508-516; Cai J, Han S, Qing R, Liao D, Law B, Boulton M E (2011) In pursuit of new anti-angiogenic therapies for cancer treatment. Front Biosci 16: 803-814). Extensive efforts have also focused on designing antibody conjugates to deliver toxic payloads in the form of cytotoxins, drug-activating enzymes, cytokines or radionuclides to tumors (Govindan S V, Goldenberg D M (2010) New antibody conjugates in cancer therapy. Scientific World Journal 10: 2070-2089). Multiple antibody engineering approaches are also being devised to improve validated therapeutics, such as trastuzumab, with the principal aims to optimize antigen specificity/affinity and effector functions of IgG antibodies (Kubota T et al (2009) Engineered therapeutic antibodies with improved effector functions. Cancer Sci 100: 1566-1572).

Antibodies of the IgE class play a central role in allergic reactions and anti-parasitic activity and have many properties that may be advantageous for cancer therapy. IgE-based active and passive immunotherapeutic approaches have been shown to be effective in both in vitro and in vivo models of cancer, suggesting the potential use of these approaches in humans (Leoh et al (2015) Curr Top Microbiol Immunol.; 388: 109-149). Thus, IgE therapeutic antibodies may offer enhanced immune surveillance and superior effector cell potency against cancer cells.

A fully human anti-HER2/neu IgE has been developed using the variable regions of the single-chain Fv C6MH3-B1 (Daniels T R et al (2012) Targeting HER2/neu with a fully human IgE to harness the allergic reaction against cancer cells. Cancer Immunol Immunother. 61: 991-1003.). C6MH3-B1 induced in vitro degranulation of RBL SX-38 cells expressing human FcεRI in the presence of murine mammary carcinoma cells expressing human HER2/neu (D2F2/E2) but not in the presence of the parental D2F2 cells that lack HER2/neu expression or shed (soluble) extracellular domain of HER2/neu (ECD^{HER2}). These results suggest that the acute inflammatory response (type I hypersensitivity) is expected to occur within the tumor microenvironment, where the HER2/neu antigen is overexpressed at high levels on the surface of cancer cells (Pegram M, Ngo D. (2006) Application and potential limitations of animal models utilized in the development of trastuzumab (Herceptin): a case study. Adv Drug Deliv Rev. 58: 723-734.), facilitating FcεRI cross-linking and triggering effector cell degranulation

A mouse/human chimeric IgE specific for the human MUC1 antigen has been developed (Teo P Z et al (2102) Using the allergic immune system to target cancer: activity of IgE antibodies specific for human CD20 and MUC1. Cancer Immunol Immunother. 61: 2295-2309.). This antibody has been shown to reduce tumor size in a murine breast carcinoma cell line expressing the transmembrane form of human MUC1 (4T1.hMUC1). However, the response was weak, possibly due to the fact that 4T1 tumors are highly avascular and grow in a densely packed mass, which may impede drug delivery or effector cell recruitment.

A novel mouse/human chimeric anti-PSA IgE composed of the variable regions of AR47.47 from PSA has also been investigated (Daniels-Wells T R et al (2013) A novel IgE antibody targeting the prostate-specific antigen as a potential prostate cancer therapy. BMC Cancer. 13: 195-207.). Priming of human dendritic cells with complexes of PSA and the anti-PSA IgE resulted in CD4 and CD8 T-cell activation in vitro. This suggests the possibility of the anti-PSA IgE to complex with PSA in the blood of patients leading to the induction of a secondary immune response involving cytotoxic T lymphocyte activity.

An alternative strategy to the passive administration of an IgE is to induce an endogenous IgE response. A novel approach to establish an active vaccination protocol is to induce tumor antigen-specific IgE through the oral route (Riemer A B et al (2007) Active induction of tumor-specific IgE antibodies by oral mimotope vaccination. Cancer Res. 67: 3406-3411). Synthetically manufactured epitope mimics (mimotopes) were generated for the epitope of human HER2/neu that is recognized by trastuzumab. The induction of high-titer serum IgE targeting the HER2/neu antigen was observed and the endogenous anti-HER2/neu IgE recognized HER2/neu-expressing human breast cancer cells (SK-BR-3), resulting in both degranulation and cytotoxicity of rat basophil cells expressing rodent FcεRI (RBL-2H3).

A mouse/human chimeric IgE antibody (MOv18 IgE), which is specific for the cancer-associated antigen folate receptor α, has been demonstrated to have superior antitumor efficacy for IgE compared with an otherwise identical IgG in a syngeneic immunocompetent animal (Karagiannis et al., Cancer Res. 2017 Jun. 1; 77(11):2779-2783). TNFα/MCP-1 signaling was identified as an IgE-mediated mechanism of monocyte and macrophage activation and recruitment to tumors.

However, it is not currently known which subjects will respond to IgE-based anti-cancer therapies. Moreover, it is not known how to promote the anti-cancer action of IgE antibodies in the most effective manner.

### SUMMARY OF THE INVENTION

Accordingly, in one aspect, the present invention provides an immunoglobulin E (IgE) for use in repolarising macrophages associated with a tumor in a subject, wherein the repolarization results in modulation of cytokine expression in the tumor microenvironment and enhanced anti-tumor activity.

In another aspect the present invention provides an immunoglobulin E (IgE) antibody for use in repolarizing macrophages from a first phenotype to an anti-tumor phenotype in the treatment of cancer in a subject; wherein the first phenotype comprises a quiescent (M0) macrophage phenotype or an anti-inflammatory (M2a) macrophage phenotype; and the anti-tumor phenotype comprises a newly polarized macrophage phenotype characterized by expression of the following cytokines and chemokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and/or interleukin-10 (IL-10).

In another aspect, the present invention provides an immunoglobulin E (IgE) antibody for use in the treatment of cancer in a subject; wherein macrophages associated with a tumor in the subject have a quiescent (M0) macrophage phenotype or an anti-inflammatory (M2a) macrophage phenotype; and the IgE treatment promotes repolarization of the macrophages associated with the tumor to a newly polarized macrophage phenotype characterized by expression of the following cytokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and/or interleukin-10 (IL-10).

In one embodiment, the newly polarized macrophages express tumor necrosis factor alpha (TNFα). In another embodiment, the newly polarized macrophages express interferon-gamma (IFNγ). In another embodiment, the newly polarized macrophages express interleukin-1beta (IL-1β). In another embodiment, the newly polarized macrophages express interleukin-6 (IL-6). In another embodiment, the newly polarized macrophages express Regulated on Activation, Normal T cell Expressed and Secreted (RANTES or CCL5). In another embodiment, the newly polarized macrophages express interleukin-10 (IL-10). In general, the newly polarized macrophage phenotype may be characterized by any combination of two or more of these cytokines. Preferably the newly polarized macrophage phenotype is characterized by expression of any three, four, five or all six of the following cytokines and chemokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10).

In one embodiment, the newly polarized macrophage phenotype is further characterized by increased expression of monocyte chemoattractant protein-1 (MCP-1), compared to an anti-inflammatory (M2a) macrophage phenotype.

In another embodiment, the newly polarized macrophage phenotype is further characterized by increased expression of interleukin-4 (IL-4), compared to an anti-inflammatory (M2a) macrophage phenotype.

In another embodiment, the newly polarized macrophage phenotype is further characterized by increased expression of interleukin-12 (IL-12), interleukin-13 (IL-13), Chemokine (C-X-C motif) Ligand 9 (CXCL9) and/or Chemokine (C-X-C motif) Ligand 11 (CXCL11) compared to a quiescent (M0) macrophage phenotype or an anti-inflammatory (M2a) macrophage phenotype.

In another embodiment, the IgE treatment further promotes increased expression of IFNγ and/or IL-12 by pro-inflammatory (M1) macrophages associated with a tumor in the subject.

In another embodiment, the quiescent (M0) macrophage phenotype or anti-inflammatory (M2a) macrophage phenotype is characterized by expression of IL-4, IL-13, MCP-1, CXCL9 and/or CXCL11.

In another embodiment, macrophages associated with a tumor in the subject are distributed around a periphery of the tumor.

In another embodiment, the newly polarized macrophage phenotype promotes further monocyte and/or macrophage recruitment into a tumor in the subject.

In another embodiment, the cancer comprises skin cancer, breast cancer, head and neck squamous cell carcinoma, prostate cancer, ovarian cancer, colon cancer, glioma, stomach cancer or pancreatic cancer.

In another embodiment, the IgE comprises an anti-folate receptor α (FRα) antibody, an anti-high molecular weight melanoma associated antigen (HMW-MAA) antibody, an anti-human epidermal growth factor receptor 2 (HER2) antibody or an anti-SF-25 antibody.

In a further aspect, the present invention provides a method for treating cancer in a subject in need thereof, the method comprising administering a therapeutically effective amount of an immunoglobulin E (IgE) to the subject, wherein macrophages associated with a tumor in the subject have a quiescent (M0) macrophage phenotype or an anti-inflammatory (M2a) macrophage phenotype; and the IgE treatment promotes repolarization of the macrophages associated with the tumor to a newly polarized macrophage phenotype characterized by expression of the following cytokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10); wherein the newly polarized macrophage phenotype has enhanced anti-tumor activity compared to the quiescent (M0) macrophage phenotype or anti-inflammatory (M2a) macrophage phenotype; thereby treating cancer in the subject.

In one embodiment, the method comprises a step of detecting one or more phenotypes of macrophages present in a tumor sample obtained from the subject; and administering the IgE to the subject if quiescent (M0) and/or inflammatory (M2a) macrophages are present in the sample at above a predetermined level.

In another embodiment, the method comprises detecting expression of one or more of the following cytokines by macrophages present in the sample: TNFα; IFNγ, IL-1β, IL-6; RANTES, IL-10, MCP-1, IL-4, IL-13, MCP-1, CXCL9, IL-12 and/or CXCL11.

### BRIEF DESCRIPTION OF THE DRAWINGS

FIG. 1 shows multiplex analysis of macrophage supernatant for TNFα, IFNγ and IL-12 cytokine profile upon IgE crosslinking. The effects of IgE cross-linking on cytokine release were investigated: cells were treated with SF-25 IgE or NIP IgE only, SF-25 or NIP IgE+anti-IgE, anti-IgE only. Values are expressed as pg/ml of supernatant±SEM, based on 3 independent experiments. NIP is 4-hydroxy-3-iodo-5-nitrophenylacetic acid.

FIG. 2 shows multiplex analysis of macrophage supernatant for IL-4, IL-10 and IL-13 cytokine profile upon IgE crosslinking. The effects of IgE cross-linking on cytokine release were investigated: cells were treated with SF-25 IgE or NIP IgE only, SF-25 or NIP IgE+anti-IgE, anti-IgE only. Values are expressed as pg/ml of supernatant±SEM, based on 3 independent experiments.

FIG. 3 shows multiplex analysis of macrophage supernatant for IL-1β, IL-6, MCP-1 and RANTES cytokine profile upon IgE crosslinking. The effects of IgE cross-linking on cytokine release were investigated: cells were treated with SF-25 IgE or NIP IgE only, SF-25 or NIP IgE+anti-IgE, anti-IgE only. Values are expressed as pg/ml of supernatant±SEM, based on 3 independent experiments.

FIG. 4 shows multiplex analysis of macrophage supernatant for MIG (CXCL9) and I-TAC (CXCL11) cytokine profile upon IgE crosslinking. The effects of IgE cross-linking on cytokine release were investigated: cells were treated with SF-25 IgE or NIP IgE only, SF-25 or NIP IgE+anti-IgE, anti-IgE only. Values are expressed as pg/ml of supernatant±SEM, based on 3 independent experiments.

### DETAILED DESCRIPTION OF THE INVENTION

It has surprisingly been found that IgE treatment produces a unique profile of cytokine and chemokine expression by different phenotypes of macrophage. In particular, it has been found that quiescent (M0) and anti-inflammatory (M2a) macrophage phenotypes can be repolarized by IgE treatment towards a novel macrophage phenotype having enhanced anti-tumor activity. This novel macrophage phenotype is referred to herein as the newly polarized macrophage phenotype. The newly polarized macrophage phenotype is characterized by a specific profile of cytokine and chemokine expression that differs from both classical pro-inflammatory (M1) and anti-inflammatory (M2a) macrophages.

The newly polarized macrophage phenotype can thus be utilized to enhance anti-cancer therapy using IgE antibodies. For instance, subjects can be selected for IgE antibody treatment based on the macrophage phenotypes that are associated with a tumor in the subject. In particular, the detection of quiescent (M0) and anti-inflammatory (M2a) macrophages in the tumor, e.g. at above a predetermined level or proportion of total macrophages, may be used as an indication that IgE therapy be used to repolarize the macrophages towards the anti-tumor phenotype.

Moreover, the present invention can be used in clinical situations where it is desirable to treat the cancer by immunotherapeutic methods, i.e. by promoting repolarizing of macrophages to the anti-tumor phenotype (e.g. to enhance the expression of the cytokines/chemokines characteristic of this phenotype) and consequently to enhance immune cell-mediated cancer cell killing by mechanisms such as ADCC. The present invention may also be used to promote further monocyte and/or macrophage recruitment into the tumor tissue, e.g. by virtue of the increased expression of chemokines such as MCP-1 by the newly polarized macrophage phenotype in addition to the tumor cells themselves. This may be particularly desirable where e.g. anti-tumor macrophages are either not present in the tumor or are present only around the periphery of the tumor tissue. The present invention thus represents a new clinical paradigm for the use of IgE therapy in treating cancer, involving the treatment of new sub-groups of patients and new clinical situations requiring repolarization of macrophages to an anti-tumor phenotype.

### Antibodies

Antibodies are polypeptide ligands comprising at least a light chain or heavy chain immunoglobulin variable region which specifically recognizes and specifically binds an epitope of an antigen, or a fragment thereof. Antibodies are typically composed of a heavy and a light chain, each of which has a variable region, termed the variable heavy (VH) region and the variable light (VL) region. Together, the VH region and the VL region are responsible for binding the antigen recognized by the antibody.

Antibodies include intact immunoglobulins and the variants and portions of antibodies well known in the art, provided that such fragments retain at least one function of IgE, e.g. are capable of binding an Fcε receptor. Antibodies also include genetically engineered forms such as chimaeric, humanized (for example, humanized antibodies with murine sequences contained in the variable regions) or human antibodies, bispecific antibodies, e.g. as described in Kuby, J., Immunology, 3rd Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are nine main isotypes or classes which determine the functional activity of an antibody molecule: IgA1-2, IgD, IgE, IgG1-4 and IgM, corresponding to the heavy chain types α, δ, ε, γ, and μ. Thus, the type of heavy chain present defines the class of antibody. Distinct heavy chains differ in size and composition; α and γ contain approximately 450 amino acids, while μ and ε have approximately 550 amino acids. The differences in the constant regions of each heavy chain type account for the different effector functions of each antibody isotype, by virtue of their selective binding to particular types of receptor (e.g. Fc receptors). Accordingly, in embodiments of the present invention the antibody preferably comprises an epsilon (ε) heavy chain, i.e. the antibody is of the isotype IgE which binds to Fcε receptors.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs has been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

Antibodies may have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). References to “VH” refer to the variable region of an immunoglobulin heavy chain. References to “VL” refer to the variable region of an immunoglobulin light chain.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimaeric antibody” comprises sequences derived from two different antibodies, which are typically derived from different species. For example, chimaeric antibodies may include human and murine antibody domains, e.g. human constant regions and murine variable regions (e.g. from a murine antibody that specifically binds to a target antigen).

Chimaeric antibodies are typically constructed by fusing variable and constant regions, e.g. by genetic engineering, from light and heavy chain immunoglobulin genes belonging to different species. For example, the variable segments of the genes from a mouse monoclonal antibody can be joined to human constant segments, such as kappa and epsilon. In one example, a therapeutic chimaeric antibody is thus a hybrid protein composed of the variable or antigen-binding domain from a mouse antibody and the constant or effector domain from a human antibody, e.g. an Fc (effector) domain from a human IgE antibody, although other mammalian species can be used, or the variable region can be produced by molecular techniques. Methods of making chimaeric antibodies are well known in the art, e.g., see U.S. Pat. No. 5,807,715.

A “humanized” antibody is an antibody including human framework regions and one or more CDRs from a non-human (for example a mouse, rat, or synthetic) antibody. The non-human immunoglobulin providing the CDRs is termed a “donor”, and the human immunoglobulin providing the framework is teamed an “acceptor”. In one embodiment, all the CDRs are from the donor immunoglobulin in a humanized immunoglobulin. The constant regions are typically substantially identical to human immunoglobulin constant regions, i.e., at least about 85-90%, such as about 95% or more identical. Hence, all parts of a humanized immunoglobulin, except the CDRs, are substantially identical to corresponding parts of natural human immunoglobulin sequences.

A humanized antibody typically comprises a humanized immunoglobulin light chain and a humanized immunoglobulin heavy chain. A humanized antibody typically binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions.

Humanized immunoglobulins can be constructed by means of genetic engineering (see for example, U.S. Pat. No. 5,585,089). Typically humanized monoclonal antibodies are produced by transferring donor antibody complementarity determining regions from heavy and light variable chains of a mouse immunoglobulin into a human variable domain, and then substituting human residues in the framework regions of the donor counterparts. The use of antibody components derived from humanized monoclonal antibodies obviates potential problems associated with the immunogenicity of the constant regions of the donor antibody. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

In embodiments of the present invention, the antibodies may be monoclonal or polyclonal antibodies, including chimaeric, humanized or fully human antibodies.

In one embodiment, the sequence of a humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in a humanized antibody framework region, are known in the art (see, for example, U.S. Pat. No. 5,585,089).

Further antibodies against a specific antigen, may also be generated by well-established methods, and at least the variable regions or CDRs from such antibodies may be used in the antibodies of the present invention (e.g. the generated antibodies may be used to donate CDR or variable region sequences into IgE acceptor sequences). Methods for synthesizing polypeptides and immunizing a host animal are well known in the art. Typically, the host animal (e.g. a mouse) is inoculated intraperitoneally with an amount of immunogen, and (in the case of monoclonal antibody production) hybridomas prepared from its lymphocytes and immortalized myeloma cells using the general somatic cell hybridization technique of Kohler, B. and Milstein, C. (1975) Nature 25 6:495-497.

Hybridomas that produce suitable antibodies may be grown in vitro or in vivo using known procedures. Monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. If desired, the antibody (monoclonal or polyclonal) of interest may be sequenced and the polynucleotide sequence may then be cloned into a vector for expression or propagation. The sequence encoding the antibody may be maintained in a vector in a host cell and the host cell can then be expanded and frozen for future use.

Phage display technology, for instance as described in U.S. Pat. No. 5,565,332 and other published documents, may be used to select and produce human antibodies and antibody fragments in vitro, from immunoglobulin variable (V) domain gene repertoires from unimmunized donors (e.g. from human subjects, including patients suffering from a relevant disorder). For example, existing antibody phage display libraries may be panned in parallel against a large collection of synthetic polypeptides. According to this technique, antibody V domain genes are cloned in frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, antibody sequences selected using phage display from human libraries may include human CDR or variable region sequences conferring specific binding to a specific antigen, which may be used to provide fully human antibodies for use in the present invention.

Methods for deriving heavy and light chain sequences from human B cell and plasma cell clones are also well known in the art and typically performed using polymerase chain reaction (PCR) techniques, examples of the methods are described in: Kuppers R, Methods Mol Biol. 2004; 271:225-38; Yoshioka M et al., BMC Biotechnol. 2011 Jul. 21; 11:75; Scheeren F A et al., PLoS ONE 2011, 6(4): e17189. doi:10.1371/journal.pone.0017189; Wrammert J et al., Nature 2008 453, 667-671; Kurosawa N et al., BMC Biotechnol. 2011 Apr. 13; 11:39; Tiller et al., J Immunol Methods. 2008 Jan. 1; 329(1-2): 112-124. Thus, antibody sequences selected using B cell clones may include human CDR or variable region sequences conferring specific binding to a target antigen, which may be used to provide fully human antibodies for use in the present invention.

### IgE Antibodies

The antibody to be administered to the subject is an IgE antibody, i.e. an antibody of the isotype IgE. There are some fundamental structural differences between IgEs and IgGs, and these have functional effects. While IgE shares the same basic molecular architecture as antibodies of other classes, the heavy chain of IgE contains one more domain than the heavy chain of IgG. The Cε3 and Cε4 domains of IgE are homologous in sequence, and similar in structure, to the Cγ2 and Cγ3 domains of IgG, so that it is the Cε2 domains that are the most obvious distinguishing feature of IgE. The Cε2 domain has been found to be folded back against the heavy chain IgE and to make extensive contact with the Cε3 domain. This bent structure of the IgE heavy chain allows it to adopt an open or closed conformation. The unbound IgE dimer has one chain in the open and one chain in the closed conformation. Binding of FcεRI to IgE is biphasic and is thought to involve initial binding to the open Cε chain followed by extensive structural rearrangement to allow binding to the closed Cε chain. The binding between the IgE dimer and the FcεRI occurs with 1:1 stoichiometry despite the presence of two identical Cε-chains. This rearrangement results in a very tight interaction between IgE and FcεRI, and a much greater affinity of IgE for its Fc receptor than found with IgG and FcγRs (McDonnell, J. M., R. Calvert, et al. (2001) Nat Struct Biol 8(5): 437-441).

The antibodies used in the present invention are typically capable of binding to Fcε receptors, e.g. to the FcεRI and/or the FcεRII receptors. Preferably the antibody is at least capable of binding to FcεRI (i.e. the high affinity Fcε receptor) or is at least capable of binding to FcεRII (CD23, the low affinity Fcε receptor). Typically, the antibodies are also capable of activating Fcε receptors, e.g. expressed on cells of the immune system, in order to initiate effector functions mediated by IgE.

The epsilon (ε) heavy chain is definitive for IgE antibodies, and comprises an N-terminal variable domain VH, and four constant domains Cε1-Cε4. As with other antibody isotypes, the variable domains confer antigen specificity and the constant domains recruit the isotype-specific effector functions.

IgE differs from the more abundant IgG isotypes, in that it is unable to fix complement and does not bind to the Fc receptors FcγRI, RII and RIII expressed on the surfaces of mononuclear cells, NK cells and neutrophils. However, IgE is capable of very specific interactions with the “high affinity” IgE receptor on a variety of immune cells such as mast cells, basophils, monocytes/macrophages, eosinophils (FcεRI, Ka. 10¹¹ M⁻¹), and with the “low affinity” receptor, Fcε RH (Ka. 10⁷ M⁻¹), also known as CD23, expressed on inflammatory and antigen presenting cells (e.g. monocytes/macrophages, platelets, dendritic cells, T and B lymphocytes).

The sites on IgE responsible for these receptor interactions have been mapped to peptide sequences on the Cε chain and are distinct. The FcεRI site lies in a cleft created by residues between Gln 301 and Arg 376 and includes the junction between the Cε2 and Cε3 domains (Helm, B. et al. (1988) Nature 331, 180183). The FcεRII binding site is located within Cε3 around residue Val 370 (Vercelli, D. et al. (1989) Nature 338, 649-651). A major difference distinguishing the two receptors is that FcεRI binds monomeric Cε, whereas FcεRII will only bind dimerised Cε, i.e. the two Cε chains must be associated. Although IgE is glycosylated in vivo, this is not necessary for its binding to FcεRI and FcεRII.

Thus, binding to Fcε receptors and related effector functions are typically mediated by the heavy chain constant domains of the antibody, in particular by domains which together form the Fc region of the antibody. The antibodies described herein typically comprise at least a portion of an IgE antibody e.g. one or more constant domains derived from an IgE, preferably a human IgE. In particular embodiments, the antibodies comprise one or more domains (derived from IgE) selected from Cε1, Cε2, Cε3 and Cε4. In one embodiment, the antibody comprises at least Cε2 and Cε3, more preferably at least Cε2, Cε3 and Cε4, preferably wherein the domains are derived from a human IgE. In one embodiment, the antibody comprises an epsilon (ε) heavy chain, preferably a human c heavy chain.

Nucleotide sequences encoding constant domains derived from human IgE, in particular Cε1, Cε2, Cε3 and Cε4 domains, are disclosed in e.g. WO 2013/050725. The amino acid sequences of other human and mammalian IgEs and domains thereof, including human Cε1, Cε2, Cε3 and Cε4 domains and human c heavy chain sequences, are known in the art and are available from public-accessible databases. For instance, databases of human immunoglobulin sequences are accessible from the International ImMunoGeneTics Information System (IMGT®) website at http://www.imgt.org. As one example, the sequences of various human IgE heavy (c) chain alleles and their individual constant domains (Cε1-4) are accessible at http://www.imgt.org/IMGT_GENE-DB/GENElect?query=2+IGHE&species=Homo+sapiens.

### Preferred Antibodies

The IgE antibodies used in the present invention may bind to any target antigen. Some non-limiting examples of such target antigens include, for instance, folate receptor α (FRα) antibody, high molecular weight melanoma associated antigen (HMW-MAA, also known as Chondroitin sulfate proteoglycan 4 or CSPG4), human epidermal growth factor receptor 2 (HER2/neu, also known as erbB2), CD20, mucin 1 (MUC1) and prostate specific antigen (PSA); i.e. the invention encompasses antibodies which bind specifically to any of the above targets or to any other antigen. Examples of suitable IgE antibodies are described in, e.g. WO2013/050725; Karagiannis et al., J Immunol 2007; 179:2832-2843; Daniels et al (2012), Cancer Immunol Immunother. 61: 991-1003; Daniels-Wells et al (2013), BMC Cancer. 13: 195-207; Teo et al (2012), Cancer Immunol Immunother. 61: 2295-2309; Karagiannis et al., Cancer Immunol Immunother. 2009 June; 58(6):915-30; and Karagiannis et al., Cancer Res; 77(11), 2017.

In general, functional fragments of the antibodies described herein may be used in the present invention. Functional fragments may be of any length as specified above (e.g. at least 50, 100, 300 or 500 nucleotides, or at least 50, 100, 200 or 300 amino acids), provided that the fragment retains the required activity when present in the antibody (e.g. specific binding to an antigen and a Fcε receptor).

Variants of the above amino acid and nucleotide sequences may also be used in the present invention, provided that the resulting antibody binds an Fcε receptor. Typically, such variants have a high degree of sequence identity with one of the sequences specified above.

The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Typically, variants may contain one or more conservative amino acid substitutions compared to the original amino acid or nucleic acid sequence. Conservative substitutions are those substitutions that do not substantially affect or decrease the affinity of an antibody to the target antigen and/or Fcε receptors. For example, a human antibody that specifically binds a target antigen may include up to 1, up to 2, up to 5, up to 10, or up to 15 conservative substitutions compared to the original sequence (e.g. as defined above) and retain specific binding to the target polypeptide. The term conservative variation also includes the use of a substituted amino acid in place of an unsubstituted parent amino acid, provided that antibody specifically binds the target antigen. Non-conservative substitutions are those that reduce an activity or binding to the target antigen and/or Fcε receptors.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### Cytokines

In embodiments of the present invention, IgE treatment may be used to promote repolarization towards a novel anti-tumor macrophage phenotype characterized by a specific profile of cytokine expression. In general, the term “cytokine” as used herein may include chemokines, which are typically small cytokines involved in promoting chemotaxis of cells such as monocytes, lymphocytes and other immune system cells.

Interferon-γ (IFN-γ) is a cytokine whose biological activity is conventionally associated with cytostatic/cytotoxic and antitumor mechanisms during cell-mediated adaptive immune response. Despite ample evidence implicating a role for IFN-γ in tumor immune surveillance, there has been a steady flow of reports suggesting that it may also have pro-tumorigenic effects under certain circumstances. The most well-characterized function of IFN-γ is the upregulation of the major histocompatibility (MHC) Class I molecules to aid in the priming and presentation of antigens in the professional antigen presenting cells (Seliger B et al (2008) IFN inducibility of major histocompatibility antigens in tumors. Adv Cancer Res. 101: 249-76). IFN-γ regulates the differentiation and function of many types of immune cells and is intimately involved in all aspects of Th1-mediated immune responses by regulating the differentiation, activation and homeostasis of T cells: it inhibits Th2 cell development, but promotes the development of regulatory T (Treg) cells (Agnello D et al (2003) Cytokines and transcription factors that regulate T helper cell differentiation: new players and new insights. J Clin Immunol. 23(3): 147-61). It also activates macrophages and induces production of chemokines. Thus, IFN-γ might be important for inducing tumor rejection.

It is known that pro-inflammatory cytokines, such as interleukin-1α (IL-1α), IL-1β, IL-6, and tumor necrosis factor α (TNFα), may either promote or suppress cancer. However, the cellular and molecular basis underlying these opposing outcomes remains enigmatic. Interleukine-1 (IL-1) is known to be up-regulated in many tumor types and has been implicated as a factor in tumor progression via the expression of metastatic and angiogenic genes and growth factors; cancer cells directly produce IL-1 or can induce cells within the tumor microenvironment to do so (Portier M et al (1993) Cytokine gene expression in human multiple myeloma. Br J Haematol. 85: 514-520). However, Haabeht et al (Oncoimmunology (2016) 5(1): e1039763) showed IL-1 (IL-1α and IL-1β) synergized with IFN-γ for the induction of tumoricidal activity in tumor-infiltrating macrophages. This synergy between IL-1 and IFN-γ may explain how inflammation, when driven by tumor-specific Th1 cells, represses rather than promotes cancer. Collectively, their data suggest a central role of inflammation and, more specifically, of the canonical pro-inflammatory cytokine IL-1 in enhancing Th1-mediated immunity against cancer.

The strong association between inflammation and cancer is reflected by high levels of IL-6 levels in the tumour microenvironment, where it promotes tumorigenesis. IL-6 is frequently viewed as a proinflammatory cytokine, with functions that parallel those of TNFα and IL-1β in the context of inflammation.

Until recently, interleukin-10 (IL-10) was regarded as an immune suppressive cytokine that hindered anti-tumor immunity. It is becoming evident that IL-10 is essential for T-helper-1 cell function and anti-tumor cytolytic activity. The potent anti-inflammatory functions of IL-10, which are mainly indirect and cell mediated, synergize with its immune stimulatory functions to improve tumor specific immune surveillance (Dennis et al (2013) Curr Opin Oncol. 25(6): 637-645).

Interleukin (IL)-4 and -13, are structurally and functionally related. They regulate immune responses and the immune microenvironment, not only under normal physiological conditions, but also in cancer. both cytokines initiate signal transduction and mediate biological effects, such as tumor proliferation, cell survival, cell adhesion and metastasis. In certain cancers, the presence of these cytokine receptors may serve as biomarkers of cancer aggressiveness. In addition, both of these cytokines and their receptors are known to play important roles in modulating the immune system for tumor growth. IL-4 causes B lymphocytes to increase and to make antibodies and also increases the production of T lymphocytes. It has been thought that IL-13 is not as critical for immune deviation as IL-4 since it cannot directly act on T cells. However, recent studies of IL-13 reveal that this cytokine plays a critical role in many aspects of immune regulation. Studies from Terabe et al (Cancer Immunol Immunother. (2004) 53(2): 79-85) and others indicate that IL-13 is central to a novel immunoregulatory pathway in which NKT cells suppress tumor immunosurveillance.

Interleukin 12 (IL-12) seemed to represent the ideal candidate for tumor immunotherapy, due to its ability to activate both innate (NK cells) and adaptive (cytotoxic T lymphocytes) immunities. While IL-12 acts on a variety of immune cells, the overall physiological role for IL-12 seems to be orchestrating the Th1-type immune response against certain pathogens. Although potent antitumor effects of IL-12 are well established, this cytokine is considered to be incapable of directly inhibiting the cancer growth, although exceptions can occur (Ferretti E et al (2010) Direct inhibition of human acute myeloid leukemia cell growth by IL-12. Immunol Lett. 133: 99-105). Rather, IL-12 acts as a major orchestrator of Th1-type immune response against cancer. Another important notion is that IL-12 appears to elicit more potent antitumor responses when existent directly in the tumor whereabouts, rather than present systemically.

Regulated on Activation Normal T Cell Expressed and Secreted (RANTES), also known as chemokine (C-C motif) ligand 5 (CCL5), is a member of the beta-family of chemokines and is a potent chemoattractant for lymphocytes and monocytes into inflammatory sites. With the help of particular cytokines (e.g. IL-2 and IFN-γ) that are released by T cells, RANTES also induces the proliferation and activation of certain natural-killer (NK) cells to form CHAK (CC-Chemokine-activated killer) cells.

Chemokine CXCL9 plays an important role in the chemotaxis of immune cells and accumulating evidence indicates that manipulation of the tumor microenvironment, which involves CXCL9, could enhance the therapeutic efficacy of strategies via tumor-specific T cells (Ding et al (2016) Cancer Med. 5(11): 3246-3259). CXCL9 could promote cancer metastasis via enhanced migration and invasion of tumor cells (Ding Q et al (2016) An alternatively spliced variant of CXCR3 mediates the metastasis of CD133+ liver cancer cells induced by CXCL9. Oncotarget. 7: 14405-14414) and breaking of the endothelial cells monolayer (Amatschek S et al (2011) CXCL9 induces chemotaxis, chemorepulsion and endothelial barrier disruption through CXCR3-mediated activation of melanoma cells. Br. J. Cancer. 104: 469-479). However, as a tumor suppressor, it has been shown mainly to recruit tumor-infiltrating CD8+ T cells and NK cells (Clancy-Thompson E et al (2015) Melanoma induces, and adenosine suppresses, CXCR3-Cognate chemokine production and T-cell infiltration of lungs bearing metastatic-like disease. Cancer Immunol. Res. 3: 956-967) and inhibit tumor angiogenesis (Addison C. L et al (2000) The CXC chemokine, monokine induced by interferon-gamma, inhibits non-small cell lung carcinoma tumor growth and metastasis. Hum. Gene Ther. 11: 247-261).

Interferon-inducible T-cell chemoattractant (I-TAC/CXCL11) is an IFN-inducible chemokine and mediates recruitment of T cells, natural killer (NK) cells and monocytes/macrophages at the site of an infection.

Nakamura et al. (in press) evaluated whether high intratumoral expression of immune mediators of the “classical” Th1, Th2, Th9 and Th17 responses may have any associations with clinical outcomes. Examination of individual mediators indicated that 9 out of the 22 mediators: CXCL-9, CXCL-10, CXCL-11, IFNγ, TNFα, IL-4, MCP-1, IL-23, CXCL-13, were significantly associated with better patient survival compared to patients with lower genomic expression. The cytokines with the most marked associations with better survival were CXCL-10 and IFNγ, associated with 22% lower risk of death (CXCL-10: HR=0.78, P=0.0037; IFNγ: HR=0.78, P=0.0036). Furthermore, subjects with higher intratumoral expression of Th2 cytokines, IL-4 and MCP-1, showed superior survival with 19% and 17% reductions in risk of death, respectively (IL-4: HR=0.78, P=0.012; MCP-1: HR=0.83, P=0.029). It is noted that the newly polarized macrophage phenotype arising from IgE crosslinking of M2a macrophages show increased expression of both IL-4 and MCP-1.

Nakamura et al also observe the greatest association with improved survival with high expression of a proportion of Th1 cytokines (IFNγ (HR=0.78, P=0.0036), CXCL-9 (HR=0.81, P=0.012), CXCL-10 (HR=0.78, P=0.0037) and CXCL-11 (HR=0.8, P=0.0078)), and also when these mediators were combined: a combination of two mediators: CXCL-9 and CXCL-10 was associated with a HR of 0.74 (P=0.0006); the combination of CXCL-9, CXCL-10 and CXCL-11 was associated with a HR of 0.73 (P=0.00028); and the highest improvement in patient survival was measured with higher expression of all four mediators (IFNγ, CXCL-9, CXCL-10, and CXCL-11) (HR=0.72, P=0.00021). These may point to synergistic functions of these mediators that may confer survival benefits. Again, it is noted that the newly polarized macrophage phenotype derived from M2a macrophages shows a similar profile with the appearance of IFNγ and an increase in the secretion of CXCL9 and CXCL11.

Nakamura et al conclude that, together, these findings identify immune secretome mediators of “classical” (Th1, Th2, and Th17) responses, part of protecting immunity to bacteria and viruses, and also those involved in pathogenic conditions such as allergies and autoimmunity, were associated with better prognosis in the context of a positive prognosis in ovarian cancer survival. Specifically, the TNFα/MCP-1 signature could have a role in anti-cancer immunity. This and other immune signatures normally deployed in infection clearance, may be enhanced by specific immunotherapy approaches such as an anti-tumour specific IgE or by attenuated parasite vaccines (Baird, J. R. et al (2013) Avirulent Toxoplasma gondii generates therapeutic antitumor immunity by reversing immunosuppression in the ovarian cancer microenvironment. Cancer Res. 73(13): 3842-51; Fox, B. A. et al (2017) Cancer therapy in a microbial bottle: Uncorking the novel biology of the protozoan Toxoplasma gondii. PLoS Pathog. 13(9): e1006523), to reverse immunosuppression and confer therapeutic benefits in ovarian and other cancers.

The cytokines and chemokines described herein and the amino acid sequences thereof (as well as nucleotide sequences encoding the amino acid sequences) are well known to a skilled person and their sequences are available from publicly accessible databases. Expression of the cytokines and chemokines by macrophage populations may be detected by any suitable method (e.g. at the polypeptide or RNA level), e.g. using (optionally labelled) antibodies directed thereto. Such antibodies are known and commercially available. For instance, the cytokines and chemokines may be detected by standard methods involving e.g. ELISA, magnetic beads and/or fluorescence-labeling. In particular, a fluorescent label (i.e. fluorophore) may be attached to an anti-cytokine antibody (or e.g. to a magnetic microparticle to which an anti-cytokine antibody is also bound). In one embodiment, cytokines may be detected using a magnetic Luminex® assay as described in the Examples. However, any other suitable may alternatively be used, including to detect expression of transcripts encoding the cytokines, e.g. RT-PCR.

### Compositions and Therapeutic Methods

Compositions are provided herein that include a carrier and one or more therapeutic IgE antibodies, or functional fragments thereof. The compositions can be prepared in unit dosage forms for administration to a subject. The amount and timing of administration are at the discretion of the treating physician to achieve the desired purposes. The antibody can be formulated for systemic or local (such as intra-tumor) administration. In one example, the therapeutic IgE antibody is formulated for parenteral administration, such as intravenous administration.

The compositions for administration can include a solution of the antibody (or a functional fragment thereof) dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical dose of the pharmaceutical composition for intravenous administration includes about 1 μg to 1 g, more preferably 10 μg to 100 mg, most preferably 0.1 to 15 mg of antibody per kg body weight of the subject per day. Dosages from 0.1 up to about 100 mg per kg per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilized form and rehydrated with sterile water before administration, although they are also provided in sterile solutions of known concentration. The antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN (Registered trademark) in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The antibody (or functional fragment thereof) can be administered to slow or inhibit the growth of cells, such as cancer cells. In these applications, a therapeutically effective amount of an antibody is administered to a subject in an amount sufficient to inhibit growth, replication or metastasis of cancer cells, or to inhibit a sign or a symptom of the cancer. In some embodiments, the antibodies are administered to a subject to inhibit or prevent the development of metastasis, or to decrease the size or number of metastases, such as micrometastases, for example micrometastases to the regional lymph nodes (Goto et al., Clin. Cancer Res. 14(11):3401-3407, 2008).

Suitable subjects may include those diagnosed with cancer, such as, but not limited to, melanoma, prostate cancer, squamous cell carcinoma (such as head and neck squamous cell carcinoma), breast cancer (including, but not limited to basal breast carcinoma, ductal carcinoma and lobular breast carcinoma), lung cancer (such as small cell lung cancer or non small cell lung cancer, including adenocarcinoma, squamous cell cancer, large cell carcinoma and mesothelioma), leukemia (such as acute myelogenous leukemia and 11g23-positive acute leukemia), lymphoma (e.g. a cutaneous lymphoma) a neural crest tumor (such as an astrocytoma, glioma or neuroblastoma), ovarian cancer, colon cancer, stomach cancer, pancreatic cancer, bone cancer (such as a chordoma), glioma or a sarcoma (such as chondrosarcoma). Preferably the antibody is administered to treat a solid tumor. In another embodiment, the antibody is administered to treat a hematological tumor (e.g. a leukemia or lymphoma).

A therapeutically effective amount of antibody will depend upon the severity of the disease and the general state of the patient's health. A therapeutically effective amount of the antibody is that which provides either subjective relief of a symptom(s) or an objectively identifiable improvement as noted by the clinician or other qualified observer. These compositions can be administered in conjunction with another chemotherapeutic agent, either simultaneously or sequentially.

In embodiments of the present invention, the therapeutic IgE antibody may be used to treat a sub-group of patients suffering from a disease such as cancer, e.g. subjects who may benefit from macrophage repolarization to an anti-tumor phenotype. Thus, the sub-group of patients to be treated may, for instance, be patients who have quiescent (M0) macrophages or anti-inflammatory (M2a) macrophages present in the tumor. The presence of such M0 or M2a macrophages in the tumor may be determined using known techniques, e.g. by detection of expression of a characteristic profile of cytokine and/or chemokines and/or cell surface markers.

The invention will now be further described by way of example only, with reference to the following non-limiting embodiments.

### Example

The role of IgE antibodies in macrophage activation and polarisation was investigated. One of the downstream effects of IgE interactions with macrophages may be the release of soluble mediators by cells upon FcεRI cross-linking (Josephs et al (2017) Anti-Folate Receptor-α IgE but not IgG Recruits 330 Macrophages to Attack Tumors via TNFα/MCP-1 Signaling. Cancer Research 77(5): 1127-1141).

For this reason, cell culture of M0, M1 and M2a macrophages were treated with SF-25 or NIP IgE and then incubated with anti-human IgE to trigger cross-linking of the FcεRI-bound antibodies. Controls of this study consisted on SF-25 or NIP IgE antibody treatment alone, anti-IgE alone and untreated cells. Supernatants were collected 4 hours after treatment to allow for cytokine production and release.

A broad panel of cytokines and chemokines was analysed, with the aim of understanding whether IgE interaction with macrophages could lead to distinct production of pro- and anti-inflammatory mediators. It was possible to derive the cytokine profiles of M0, M1 and M2a macrophages by performing a multiplex assay with magnetic beads, which allows the simultaneous detection of many different soluble mediators within the same sample.

### Methods and Materials

### Primary Cell Isolation and Culture

K2EDTA-spray coated collection tubes were used to collect peripheral venous blood (50 ml) from healthy donors. To isolate peripheral blood mononuclear cells (PBMCs), equal volumes of blood and 2% FCS/2 mM EDTA were gently mixed to a final volume of 30 ml and gently pipetted on top of 15 ml of Ficoll-Paque™ PLUS density gradient in a 50 ml conical tube. The tube was then centrifuged at 1200×g with slow acceleration and no brake at room temperature (RT) for 20 minutes. The plasma interface was harvested using a plastic Pasteur pipette, transferred in a new vial and washed with PBS at 600×g with maximum acceleration and deceleration at 4° C. for 10 minutes. The erythrocytes present in the sample were lysed by incubating the cells with 5 ml of RBC lysis buffer for 5 minutes at RT, followed by washing with PBS+2% FCS/2 mM EDTA.

Monocytes were isolated from human blood using the Pan Monocyte Isolation Kit (Miltenyi Biotec), an indirect magnetic labelling system for the isolation of untouched monocytes from human PBMCs. Taking advantage of this technique, the simultaneous enrichment of classical (CD14++CD16++), non-classical (CD14+CD16++) and intermediate (CD14++CD16+) monocyte populations was performed. A highly pure monocyte population is obtained by depletion of the labelled and magnetically-bound cells. Once the PBMCs were isolated, the cells were passed through a 40 μm cell strainer to remove any clumps and the number of cells in the sample was determined using a haemocytometer. Next, 1×10⁸ PBMCs were transferred in a new vial and washed with PBS at 600×g for 5 minutes with maximum acceleration and deceleration. After discarding the supernatant, cells were resuspended in 400 μl of MACS buffer (PBS supplemented with 0.5% Fetal Bovine Serum and 2 mM EDTA).

To isolate human monocytes by negative selection, the Pan Monocyte Isolation Kit (Miltenyi Biotec) protocol was followed. Briefly, 100 μl of FcR Blocking Reagent, was added to the sample to block unwanted binding of antibodies to human Fc receptors and after pipetting up and down, 100 μl of Biotin-Antibody Cocktail was added and in order to promote the antibody binding to monocytes, the sample was incubated at 4° C. for 5 minutes. After this, 300 μl of MACS buffer and 200 μl of Anti-Biotin Microbeads, were added to the vial by mixing well. Cells were then incubated again at 4° C. for 10 minutes. After the incubation the sample was subject to magnetic cell separation by inserting a LS column into a MidiMACS™ Separator mounted onto a Multi Stand (Miltenyi Biotec). The column was first rinsed with 3 ml of MACS buffer and when the reservoir was empty, the cell suspension was applied to the column. The flow-through, representing the enriched monocyte fraction, was collected. The column was washed by loading 3 ml of MACS buffer 3 times and the unlabeled cells were collected and combined with the effluent collected the step before. To collect the labelled cells, representing mainly lymphocytes, the column was removed from the separator and placed onto a suitable collection tube. Five ml of MACS buffer were loaded onto the column and, using the plunger, the magnetically labelled non-monocytes were flushed out.

To check the level of purity of the monocyte population, the sample was prepared for flow cytometry (performed using a BD FACSCanto™ II at the Biomedical Research Centre Flow Cytometry Core—King's College London) as follows: cells were stained with a BV786-conjugated mAb recognising CD14 (1 μl per 1×10⁵ cells) and a BV711-conjugated mAb against CD16 at 4° C. for 30 minutes. After incubation cells were washed once in FACS buffer (Phosphate Buffer Saline (PBS; Gibco) supplemented with 2% Fetal Calf Serum (Gibco)) and maintained in fresh FACS buffer until ready for acquisition at the flow cytometer. For each fluorophore, single positive and FMO controls were used to set up the gates.

Once monocytes were isolated through magnetic separation mentioned above, they were resuspended in RPMI 1640 medium GlutaMAX (Gibco), 2% FBS to promote adhesion and cells were seeded in 6-well plates at a density of 1-1.5×10⁶ cells/ml; each well was loaded with 2 ml of cell suspension and plates were placed in a tissue culture incubator. After 2 hours of incubation, adhesion of monocytes was checked under the microscope and media were carefully removed by tilting the plate and pipetting it out. To remove cells that were not adhered completely, each well was washed with 1 ml of sterile PBS and replenished with 2 ml of RPMI 1640 supplemented with 10% FCS and penicillin/streptomycin (Penicillin (100 U/ml) and Streptomycin (100 U/ml) (Life Technologies)) with the addition of 20 ng/ml of M-CSF (Monocyte Colony Stimulating Factor; Peprotech) to allow monocyte to grow ex-vivo. Thereafter, every 3 days, half of the volume in each well fresh were replaced with media containing 40 ng/ml M-CSF. After 7 days differentiated macrophages were observed.

From this point onwards, different cytokines were added to the cell culture based on the desired cell phenotype: M1 or M2 macrophages. To polarize macrophages towards an M1 phenotype, 20 ng/ml of Interferon-Gamma (IFNγ; Life Technologies) and 100 ng/ml of Lipopolysaccharide (LPS; Sigma) was added to the cells for 72 hours. On the other hand, to polarize macrophages towards an M2 phenotype, 20 ng/ml of Interleukin-4 (IL-4; Peprotech) was added for 72 hours.

### SF-25 IgE and IgG1 Antibody Production

SF-25 IgE Production with Spinner Bottles:

Sp2/0 cells expressing SF-25 IgE antibody were seeded in a 1 L spinner bottle with a total initial volume of 180 ml of IMDM medium at a cell density of 5×105 cells/ml. The suspension was then topped up to 500 ml with fresh medium and placed on spinner plates inside a humidified incubator at 37° C. and 5% CO2 with a constant speed of 7.5 rpm. After 3 days, fresh medium was added to cell suspensions up to 1 L of final volume, and the cells were left growing with no more fresh medium addition for 3 weeks. Every 3 days, a 1 ml sample was collected from each cell suspension in order to monitor the antibody production over time. The sample was collected in a 1.5 ml tube, centrifuged at 12500×g for 5 minutes, filter-sterilised with a 0.45 μm filter and stored at −20° C.

SF-25 IgE Production with Shaker Flasks:

Sp2/0 cells expressing SF-25 IgE antibody were seeded in a 1 L Erlenmeyer flask with an initial volume of 60 ml of IMDM complete medium and a cell density of 5×105 cells/ml. The suspension was then topped up to 150 ml with fresh complete medium and placed in a humidified incubator at 37° C. and 5% CO2 on a shaker platform with a constant speed of 55 rpm. Every 3 days, a 1 ml sample was collected from the cell suspension in order to monitor the antibody production over time. The sample was collected in a 1.5 ml tube, centrifuged at 12500×g for 5 minutes, filter-sterilised with a 0.45 μm filter and stored at −20° C.

### SF-25 IgE Production in Roller Bottles

Sp2/0 cells expressing SF-25 IgE antibody were seeded in a 2 L Roller Bottle with a density of 5×105 cells/ml in a total volume of 250 ml IMDM complete medium. Cells were then given CO2 infusion by the insertion of a sterile 10 ml pipette into the cell suspension: the pipette was then connected to a CO2 tank and gas was let to flow inside the cell culture with a maximum pressure of 1.5 mBar for 2 minutes. Once the pipette was removed, the lid of the bottle was closed firmly, and the bottle was placed on a rotating cylinder at 37° C. Every 3 days, the cell density was monitored and cell cultures were diluted with fresh medium 1:2 until the maximum working volume of 1200 ml was reached. Cells were given CO2 every three days as described previously or at the end of a two-week culture, at which point cell supernatants were harvested.

### SF-25 IgE Antibody Purification

Following production of antibody in Sp2/0 cell supernatants, in order to purify SF-25 IgE antibody, cell supernatants were centrifuged at 400×g for 30 minutes to pellet the cells. After centrifugation, the supernatant was collected and filtered using Stericup® 0.45 μm Filter Units. Supernatants were given a 0.01% of sodium azide before being applied to affinity chromatography. Purification of human chimeric SF-25 IgE was performed using a HiTrap KappaSelect column (GE Healthcare Life Sciences).

All affinity chromatography purifications of SF-25 IgE were performed using an automated apparatus fitted with a peristaltic pump (ÄKTA Prime), passing fluid over the column at 1 ml/min. The first steps involved washing the column with PBS, then passing the cell culture supernatant containing secreted antibody over the column to bind to the agarose beads. Captured antibody was then eluted with an elution buffer (0.2 M Glycine, pH 2.3). The eluate was immediately neutralized with neutralization buffer (1 M Tris, pH 8.2). The column was then re-equilibrated by cycling of PBS solution. In order to exclude any aggregated and/or degradation antibody products, high performance liquid chromatography (HPLC) size-exclusion chromatography was performed. The entire process was carried out at 4° C.

### Macrophage Activation Via IgE Antibodies

### IgE Cross-Linking on Monocyte-Derived-Macrophages (MDM)

In order to understand which role IgE antibodies may play in macrophage polarization and activation, engagement and cross-linking experiments of IgE on the surface of monocyte-derived macrophages were carried out, using SF-25 and NIP IgE antibodies as described in e.g. EP 0397700B1 and Gould, H. J et al., Eur. J. Immunol. 1999. 29: 3527-3537. NIP IgE is a non-specific IgE raised against the hapten nitrohydroxyiodophenylacetate (4-hydroxy-3-iodo-5-nitrophenylacetic acid).

M0, M1 and M2a (MDM) macrophages were incubated with 5 μg/ml of IgE antibody (Dako) for 1 hour at 37° C. by adding the antibody solution directly onto the cell culture plate. After 1 hour, supernatants were completely removed with a sterile pipette, 1 ml of sterile PBS was added to each well and then removed to wash any unbound antibody away. Where applicable, cells were then treated with 1 μg/ml of polyclonal anti-human IgE antibody to cross-link the IgE antibodies already bound to the cell surface. Cells were incubated for one additional hour at 37° C.

Cells were harvested 4 hours after stimulation to examine MDM cell surface marker expression and supernatants collected 24 h after stimulation to investigate cytokine release.

### Magnetic Luminex Assay of Macrophage Supernatants for Cytokine Detection

Macrophage supernatants were collected 4 hours post-stimulation to allow cells to produce and secrete cytokines under various stimulation conditions. Supernatants were harvested by gently tilting the culture plate and pipetted out without disturbing the adherent cells. Samples were then centrifuged at 600×g to remove floating cells and debris and then transferred in clean tubes for storage at −20° C. until use.

On the day of the assay, samples were thawed at room temperature, diluted 1:1 with sterile PBS and kept on ice until ready to be loaded onto the assay plate.

The multiplex assay was performed with the Magnetic Luminex Kit following the protocol instructions. Briefly, 50 μl of the microparticle cocktail were added to each well of the plate, immediately followed by the addition of 50 μl of sample. The plate was sealed and incubated at room temperature for 2 hours on an orbital shaker. Washes were performed by adding and removing 100 μl of wash buffer to each well. The wash procedure was repeated 3 times. Once the liquid of the last wash was removed, each well was loaded with 50 μl of diluted Biotin Antibody Cocktail, the plate was sealed and incubated at room temperature for 1 hour on an orbital shaker. Wash step was repeated as above and 50 μl of Streptavidin-PE were added to the wells. The plate was sealed again and incubated as above for 30 minutes. After having performed an additional wash step, the microparticles were resuspended by adding 100 μl of Wash Buffer to each well, the plate was sealed and left on the shaker up to 90 minutes until ready to be analysed on a Luminex™ analyzer.

### Results

### Cytokines Involved in Th1 Response—IL-12, IFNγ, TNFα

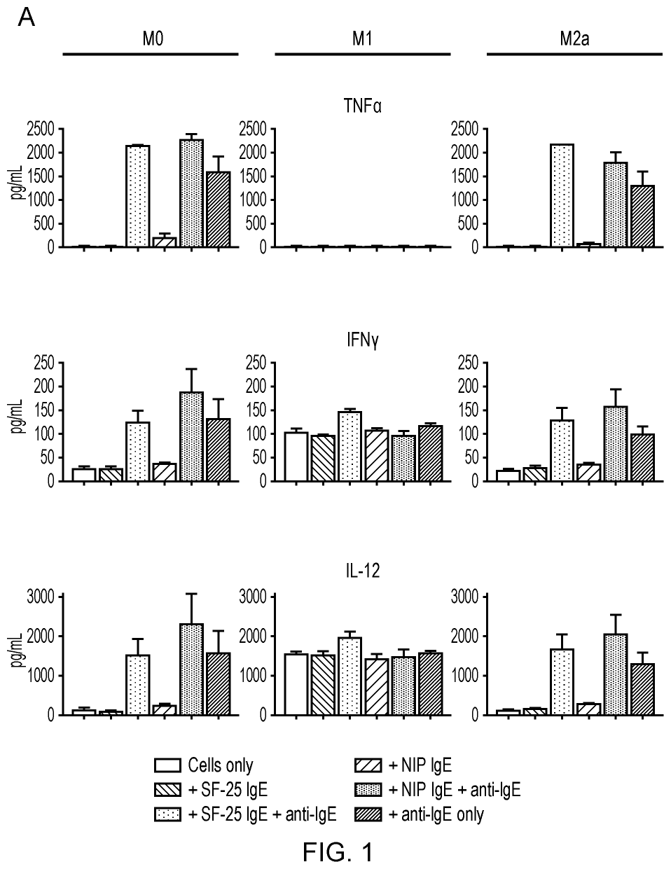

Cross-linking of IgE led to a drastic increase (from 100 to approximately 2,000 pg/ml) in TNFα secretion in both M0 and M2a macrophages, while no or very low production of the mediator was detected in M1 cells in all the experimental conditions tested (FIG. 1).

Although of lower magnitude, IgE cross-linking enhanced the production of IFNγ by both M0 and M2a subsets. M1 macrophages showed a 4-fold higher baseline level of IFNγ compared to the other subsets, but concentration of IFNα remained stable upon activation via IgE.

The same type and magnitude of modulation was observed when IL-12 concentration was tested: M0 and M2a macrophages increased IL-12 production from 100 to approximately 1500-2000 pg/ml when cross-linked with both SF-25 and NIP IgE antibodies. M1 cells featured a greater baseline level of IL-12 (ca. 1,500 pg/ml), which remained unaffected upon IgE treatment.

### Anti-Inflammatory Cytokines—IL-4, IL-10, IL-13

IL-4 production slightly increased upon IgE cross-linking in both M0 and M2a subsets but remained unaffected in M1 cells.

With regards to IL-10, baseline levels were very similar between M0 and M1 macrophages (20 and 25 pg/ml) and much lower (ca. 7 pg/ml) in M2a. IgE cross-linking, though, effectively upregulated the release of IL-10 in M0 and M2a subsets, while levels remained unchanged in M1 macrophages.

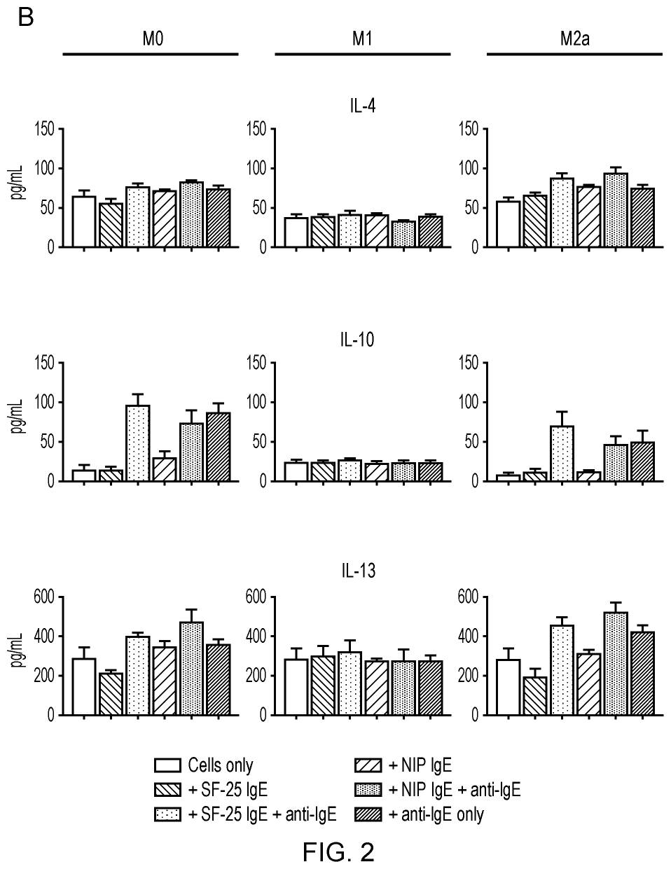

The background concentration of IL-13 was the same across all three macrophage populations. However, in M0 and M2a cells, but not in M1 macrophages, cross-linking of IgE increased IL-13 secretion after cross-linking of SF-25 and NIP IgE on the cell surface (FIG. 2).

### Cytokines Involved in Inflammatory Response—IL-1γ and IL-6

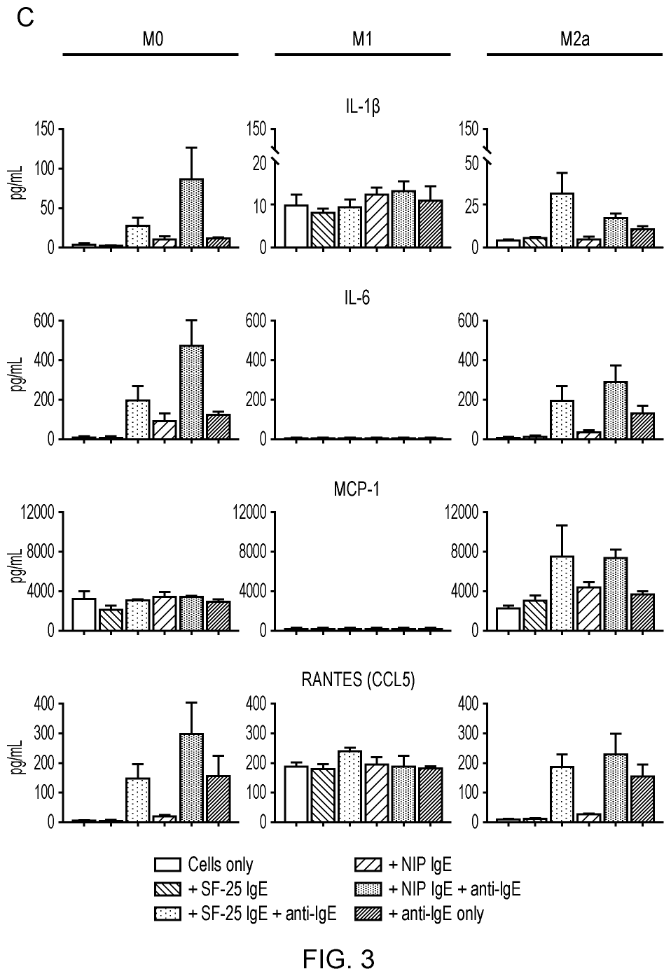

Once again, the response to IgE activation was similar between M0 and M2a phenotypes when analysing both IL-1β and IL-6 secretion. Baseline concentration of both cytokines appeared very low across the three cell subsets and remained stable across all treatments in M1 macrophages. However, cross-linking of cell-bound IgE caused a net increase in IL-1β and IL-6 secretion in M0 and M2a macrophages (FIG. 3).

### Macrophage Chemoattractant Chemokines—MCP-1 (CCL2) and RANTES (CCL5)

MCP-1 is known to recruit monocytes, macrophages and dendritic cells to the site of inflammation. This action to the inflammatory site is also promoted by RANTES, a modulator of many important macrophage functions like chemotaxis and phagocytosis. MCP-1 and RANTES concentration upon IgE activation was therefore tested here, since any modulation of secretion would help further dissect the interaction of this antibody class with macrophages.

Cross-linking of cell-bound IgE produced no modulation of MCP-1 supernatant concentration in either M0 or M1 macrophages. The M2a subset, on the other hand, responded to IgE activation by enhancing the release of MCP-1 in all conditions tested.

Modulation of RANTES secretion upon IgE treatment showed the same pattern in both M0 and M2a macrophages, where we detected a drastic increase in its production when cell-bound IgE were cross-linked. Interestingly, when RANTES levels were increased in M0 and M2a macrophages, the peak concentration corresponded approximately to the baseline concentration in M1 cells, where instead no modulation of RANTES secretion was detected across all conditions tested (FIG. 3).

### Interferon Gamma-Induced Chemokines—MIG (CXCL9) and I-TAC (CXCL11)

MIG concentration in M1 macrophages remained unchanged amongst the different conditions tested, indicating that IgE activation does not affect its production in this subset of macrophages. Similar effects were observed between M0 and M2a macrophages instead, where cross-linking of IgE led to a slight increase in MIG secretion.

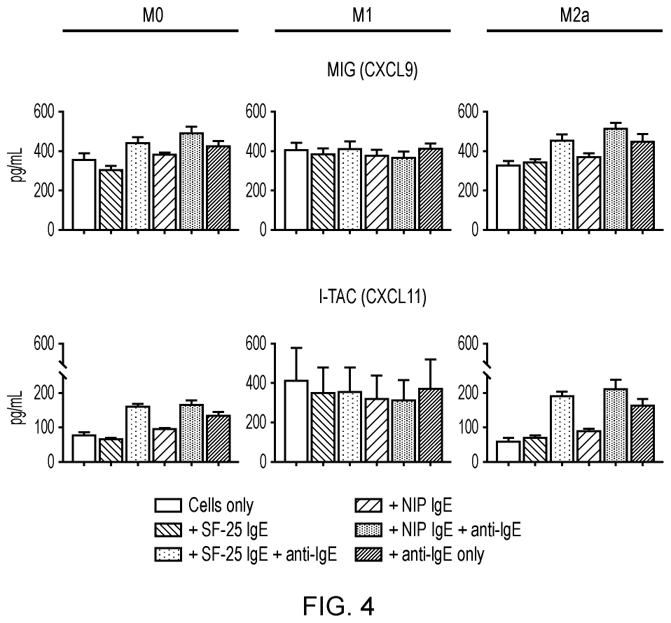

Analysis of I-TAC concentration revealed that the background level of this chemokine was much higher in M1 macrophages compared to the other two subsets: M1 featured a baseline concentration of ca. 400 pg/ml, approximately four times greater than that detected on M0 and M2a cells. Despite this, treatment of M0 and M2a cultures with IgE cross-linking drastically increased the amount of I-TAC released in the supernatant, increasing its concentration from ca. 80 to ca. 160 pg/ml in M0 cells and from 50 to ca. 200 pg/ml in M2a macrophages (FIG. 4).

In conclusion, it was observed that for all soluble mediators tested but from MCP-1, the cross-linking of cell-bound IgE triggered the same effect on M0 and M2a macrophages. IgE cross-linking on M2a macrophages but not on M0 or M1 subsets, triggered enhanced secretion of MCP-1. On the other hand, M1 macrophages appeared fairly unresponsive upon IgE activation with two notable exceptions: IgE cross-linking triggered upregulation of the pro-inflammatory mediators IFNγ and IL-12 (Table 3).

TABLE 3
Soluble mediator production modulation in macrophages upon
cell-bound IgE cross-linking

| | Secreted Cytokines/Chemokines | | |
|---|---|---|---|
| | Physiological state/ | IgE | Final macrophage |
| | IgE engagement | Crosslinking | phenotype |
| M0 | IL-4 | TNFα | Newly polarized |
| | IL-13 | IFNγ | macrophage |
| | MCP-1 | IL-1β | phenotype |
| | CXCL9 | IL-6 | |
| | CXCL11 | RANTES | |
| | | IL-10 | |
| | | ↑IL-12 | |
| | | ↑IL-13 | |
| | | ↑CXCL9 | |
| | | ↑CXCL11 | |
| M1 | IFNγ | ↑IFNγ | M1 |
| | IL-12 | ↑IL-12 | |
| | IL-13 | | |
| | RANTES | | |
| | CXCL9 | | |
| | CXCL11 | | |
| M2a | IL-4 | TNFα | Newly polarized |
| | IL-13 | IFNγ | macrophage |
| | MCP-1 | IL-1β | phenotype - |
| | CXCL9 | IL-6 | The only phenotype |
| | CXCL11 | RANTES | with augmented |
| | | IL-10 | MCP-1 secretion |
| | | ↑IL-4 | after IgE cross- |
| | | ↑IL-12 | linking |
| | | ↑IL-13 | |
| | | ↑MCP-1 | |
| | | ↑CXCL9 | |
| | | ↑CXCL11 | |

Table 3 sets out changes in cytokine and chemokine release in macrophages before and after IgE-crosslinking: ↑ increased detection, ↓ decreased detection, when a soluble mediator is not repeated from the column entitled: “Physiological state” to column named: “IgE Crosslinking”, its production remained unmodified between the two conditions. Crosslinking of cell-bound IgE on M0 and M2a macrophages either retained or increased the production of all soluble mediators tested. Interestingly, MCP-1 was upregulated only upon cross-linking of M2a cells, suggesting a potential role of anti-tumor IgE antibodies in inducing MCP-1 production and secretion by M2a macrophages. The polarisation status of M1 macrophages was retained upon stimulation of cells with IgE, suggesting that IgE antibodies might keep a pro-inflammatory profile in the TME. In contrast, cross-linking of cell-bound IgE on M0 and M2a macrophages led to a newly polarised macrophage phenotype, secreting both pro- and anti-inflammatory mediators, therefore describing the remarkable effect of IgE antibodies in skewing a quiescent (M0) or pro-tumour (M2a) phenotype to a new one featuring new pro-inflammatory activities.

Macrophages are tissue-resident phagocytes and antigen-presenting cells (APC) which differentiate from circulating peripheral blood monocytes. They perform important active and regulatory functions in innate as well as adaptive immunity (Murray P J, Wynn T A (2011). Protective and pathogenic functions of macrophage subsets. Nat Rev Immunol, 11(11): 723-37). Activated macrophages of different phenotypes are routinely classified into M1-macrophages (CAM) and M2-macrophages (AAM). The classically activated M1-macrophages comprise immune effector cells with an acute inflammatory phenotype. These are highly aggressive against bacteria and produce large amounts of lymphokines (Murray P J, Wynn T A (2011). Obstacles and opportunities for understanding macrophage polarization. J Leukoc Biol, 89(4):557-63). The alternatively activated, anti-inflammatory M2-macrophages can be separated into at least three subgroups. These subtypes have various different functions, including regulation of immunity, maintenance of tolerance and tissue repair/wound healing. Indeed, cells of the monocyte/macrophage lineage exhibit extraordinary plasticity in response to endogenous as well as exogenous stimuli, which can allow overriding of the initial M1/M2-polarization processes, for example M2-polarized macrophages can convert to the M1-activated status under certain conditions.

Primary human macrophages are difficult to isolate in sufficient amounts from tissue and do not proliferate in culture. In addition, it is commonly accepted that the obtained cells often exhibit significant phenotypical heterogeneity. Monocyte-derived Macrophages (MDM) provide an excellent alternative, since human blood monocytes are readily available in large numbers and can be differentiated into macrophages in vitro.

Most work on characterising macrophages has been carried out on murine cells, which differ considerably in profile to human-derived macrophages. For example, CD206 is a good M2 marker in mice but CD200R seems to work better in human cells. In addition, the differences between M1 and M2 markers tend to be quantitative. For example, both M1 and M2 will express MHCII, but M1 will express it with much greater intensity. The same applies to most if not all surface markers that are used for M1/M2 differentiation, so a “positive control” is highly desirable. However, a commonly accepted marker profile for M1-macrophages is CD68+/CD80+/CD163− or CD163 low, whereas M2-macrophages are characterized as CD68+/CD80−/CD163+. However, CD163 upregulation depends on how the macrophages are polarized (IL-4/IL-10, RPMI medium or ex-vivo). Ideally, multiple approaches are used for characterisation, including morphology (M1: egg shape or round; M2: fried egg shape or with dendrites), gene expression (e.g. iNOS/Arg1 ratio, cytokine IL-1beta, chemokine CCL2 and Ptgs2), and flowcytometric analysis.

### Discussion

Monoclonal antibodies (mAbs) can exert anti-tumour effects through a multitude of mechanisms. However, it has recently emerged how mAbs acting via Fc-mediated mechanisms play a key role in engaging the immune system and modulating the immune profile of the tumour microenvironment (Bakema et al (2014) Fc Receptor-Dependent Mechanisms of Monoclonal Antibody Therapy of Cancer. In Current topics in microbiology and immunology, 382: 373-392); Moore et al (2010) Engineered Fc variant antibodies with enhanced ability to recruit complement and mediate effector functions. mAbs 2(2): 181-189). A number of mAbs currently in use, such as Trastuzumab and Ipilimumab, have provided evidence of the substantial contributions that Fc-mediated mechanisms can confer to the clinical efficacy of these class of anti-cancer agents (Peggs et al, J Exp Med 2009 Aug. 3; 206(8):1717-1725; Simpson et al. J Exp Med 2013 Aug. 26; 210(9):1695-1710; Romano et al. Proc Natl Acad Sci USA 2015 May 12; 112(19):6140-6145; Arce Vargas et al (2018) Fc Effector Function Contributes to the Activity of Human Anti-CTLA-4 Antibodies. Cancer Cell. 33(4): 649-663.e4; Shi et al (2014) Engagement of immune effector cells by trastuzumab induces HER2/ERBB2 downregulation in cancer cells through STAT1 activation. Breast Cancer Research. 16(2): R33). Particular attention has therefore been dedicated to Fc engineering, in order to modulate not only the cytotoxic functions of mAbs, but also to overcome the immunosuppressive microenvironment in the majority of solid malignancies (Bakema et al (supra); Dahan et al (2015) FcγRs Modulate the Anti-tumor Activity of Antibodies Targeting the PD-1/PD-L1 Axis. Cancer Cell. 28(3): 285-295; Moore et al (supra)). Amongst the strategies explored to engineer the Fc domain to improve its clinical efficacy, the modification of its glycosylation profile and the selection of different isotype subclasses represent the ones investigated the most (Ferrara et al (2006) Modulation of therapeutic antibody effector functions by glycosylation engineering: Influence of Golgi enzyme localization domain and co-expression of heterologous β1, 4-N-acetylglucosaminyltransferase III and Golgi α-mannosidase II. Biotechnology and Bioengineering. 93(5): 851-861; Kellner et al (2017) Modulating Cytotoxic Effector Functions by Fc Engineering to Improve Cancer Therapy. Transfusion Medicine and Hemotherapy: Offizielles Organ Der Deutschen Gesellschaft Fur Transfusionsmedizin Und Immunhamatologie. 44(5): 327-336; Schlothauer et al (2016) Novel human IgG1 and IgG4 Fc-engineered antibodies with completely abolished immune effector functions. Protein Engineering Design and Selection. 29(10): 457-466).

In contrast, one of the strategies that remains poorly investigated is the employment of antibodies with Fc regions of a distinct class from the commonly used IgG. Engineering antibodies of the IgE class represents one such strategy. In fact, since different Fc-receptors are expressed by distinct immune cell subsets, the employment of a new antibody isotype like IgE would translate to the engagement of a diverse panel of immune effector cells and, ultimately, to a potentially improved clinical outcome.

The potential advantages of IgE antibodies in the treatment of solid malignancies rely upon the unique biological properties of this immunoglobulin class, alongside the demonstrated infiltration in the tumor microenvironment of many key IgE receptor-expressing immune effector cells. Based on these findings, a number of IgE-based immunotherapeutic approaches including recombinant IgE antibodies targeting tumour antigens were developed, with the aim of triggering IgE-mediated immune responses against tumour cells (Daniels et al (2012) Targeting HER2/neu with a fully human IgE to harness the allergic reaction against cancer cells. Cancer Immunology, Immunotherapy. 61(7): 991-1003; Josephs et al (2017) Anti-Folate Receptor-α IgE but not IgG Recruits 330 Macrophages to Attack Tumors via TNFα/MCP-1 Signaling. Cancer Research. 77(5): 1127-1141; Josephs et al (2018) An immunologically relevant rodent model demonstrates safety of therapy using a tumour-specific IgE. Allergy. 2018 December; 73(12):2328-2341; Karagiannis et al (2012) Recombinant IgE antibodies for passive immunotherapy of solid tumours: from concept towards clinical application. Cancer Immunology, Immunotherapy: CII, 61(9), 1547-1564; Kershaw et al (1998) Tumor-specific IgE-mediated inhibition of human colorectal carcinoma xenograft growth. Oncology Research. 10(3): 133-142).

The cytokine and chemokine analysis performed on supernatants collected from macrophage cell cultures stimulated with IgE allowed the identification of soluble mediators upon either IgE engagement along or IgE cross-linking.

In M1 macrophages, IgE cross-linking retained the production of pro-inflammatory cytokines such as IFNγ and IL-12. Josephs et al recently demonstrated upregulation of pro-inflammatory immune-associated pathways including IL-12 and NK-cell immune activation signatures in the lungs of tumour-bearing rats which were treated with MOv18 IgE (Josephs et al., 2018 supra). Considering these findings, it is possible that IgE engagement and cross-linking may at least retain M1 macrophages in a manner consistent with known pro-inflammatory and antigen-presenting functions of this subset.

The secretion of all mediators tested except one (IL-1β, IL-4, IL-6, IL-10, IL-12, IL-13, IFNγ, TNFα, RANTES, MIG and I-TAC) was modulated in the same manner by cross-linking of cell-bound IgE on non-activated M0 and M2a macrophages. This suggests that the two subsets might feature similar molecular mechanisms in their ability to regulate the activation of the FcεRI pathway. More specifically, the production of all soluble mediators tested was either retained at the same level or increased with IgE-cross-linking. The cytokine panel secreted by M0 and M2a after cross-linking with SF-25 IgE did not correspond to a defined macrophage subtype. Yet, based on the mediator signature displayed by IgE-stimulated cells, this might represent a newly polarised macrophage subset secreting both pro- and anti-inflammatory mediators, as well as chemoattractant factors. IgE cross-linking on both M0 and M2a macrophages triggered enhanced levels of the pro-inflammatory M1 cytokine TNFα. On the other hand, IgE cross-linking had different effects in M0 and M2a macrophages in the relation to the production of the macrophage chemoattractant MCP-1. M2a macrophages, upon IgE treatment, enhanced the production and the release of MCP-1.

Chemokines such as monocyte chemoattractant protein-1 (MCP-1) bind to specific cell surface transmembrane receptors coupled with heterotrimeric G proteins, whose activation leads to the activation of intracellular signalling cascades that prompt migration toward the chemokine source. MCP-1, also referred to as CCL2, regulates the migration and infiltration of monocytes, memory T lymphocytes, and natural killer (NK) cells. Monocytes are critical for the initiation of tumor arteriogenesis because they adhere to and invade endothelium activated by the increased shear stress that results from large pressure differences between perfused areas (Scholz et al (2001) Arteriogenesis, a new concept of vascular adaptation in occlusive disease. Angiogenesis. 4: 247-257). MCP-1 is implicated in this process because it not only attracts monocytes, but also promotes their adhesion by inducing them to upregulate MAC-1, the receptor for intracellular adhesion molecule-1 (ICAM-1) that is expressed in activated endothelium. MCP-1 has also been shown to augment cytostatic activity against tumor cells upon addition to macrophages in tissue culture (Zachariae et al (1990) Properties of monocyte chemotactic and activating factor (MCAF) purified from a human fibrosarcoma cell line. J Exp Med. 171: 2177-2182), thus driving cells to apoptosis.

A growing body of epidemiological and clinical data supports the concept that chronic inflammation promotes tumor development and progression. As a major pro-inflammatory cytokine, tumor necrosis factor (TNFα) is able to act as an endogenous tumor promoter to bridge inflammation and carcinogenesis: TNFα is known to stimulate proliferation, survival, migration, and angiogenesis in most cancer cells that are resistant to TNF-induced cytotoxicity, resulting in tumor promotion. However, TNFα also has a capacity to suppress tumor cell proliferation and induce tumor regression. Thus, TNFα is a double-edged sword that could be either pro- or anti-tumorigenic (Wang and Lin (2008) Tumor necrosis factor and cancer, buddies or foes? Acta Pharmacol Sin. 29(11): 1275-1288). Numerous agents, including naturally occurring and synthetic compounds, have been shown to sensitize tumor cells to TNFα-induced cell death through inhibiting NF-κB activation. Combining these compounds with TNFα led to synergistic cytotoxicity in tumor cells [Wang X et al (2006) 17-allylamino-17-demethoxygeldanamycin synergistically potentiates tumor necrosis factor-induced lung cancer cell death by blocking the nuclear factor-kappaB pathway. Cancer Res. 66(2): 1089-95; Zhang S et al (2004) Suppressed NF-kappaB and sustained JNK activation contribute to the sensitization effect of parthenolide to TNF-alpha-induced apoptosis in human cancer cells. Carcinogenesis. 25(11): 2191-9; Fas S C et al (2006) Wogonin sensitizes resistant malignant cells to TNFalpha- and TRAIL-induced apoptosis. Blood. 108(12): 3700-6; Ju W et al (2007) A critical role of luteolin-induced reactive oxygen species in blockage of tumor necrosis factor-activated nuclear factor-kappaB pathway and sensitization of apoptosis in lung cancer cells. Mol Pharmacol. 71(5): 1381-8; Rae C et al (2007) Elevated NF-kappaB responses and FLIP levels in leukemic but not normal lymphocytes: reduction by salicylate allows TNF-induced apoptosis. Proc Natl Acad Sci USA. 104(31): 12790-5; Shukla S, Gupta S (2004) Suppression of constitutive and tumor necrosis factor alpha-induced nuclear factor (NF)-kappaB activation and induction of apoptosis by apigenin in human prostate carcinoma PC-3 cells: correlation with down-regulation of NF-kappaB-responsive genes. Clin Cancer Res. 10(9): 3169-78; Shishodia S et al (2006) A synthetic triterpenoid, CDDO-Me, inhibits IkappaBalpha kinase and enhances apoptosis induced by TNF and chemotherapeutic agents through down-regulation of expression of nuclear factor kappaB-regulated gene products in human leukemic cells. Clin Cancer Res. 12(6): 1828-38). In addition, TNFα can be used as an adjuvant reagent to promote the anti-cancer effect of chemotherapy agents such as doxorubicin (Cao W et al (2005) TNF-alpha promotes Doxorubicin-induced cell apoptosis and anti-cancer effect through downregulation of p21 in p53-deficient tumor cells. Biochem Biophys Res Commun. 330(4): 1034-40), sensitize low epidermal growth factor receptor (EGFR)-expressing carcinomas to anti-EGFR therapy (Hambek M et al (2001) Tumor necrosis factor alpha sensitizes low epidermal growth factor receptor (EGFR)-expressing carcinomas for anti-EGFR therapy. Cancer Res. 61(3): 1045-9), or overcome acquired resistance to EGFR tyrosine kinase inhibitor in non-small-cell lung cancer cells (Ando K et al (2005) Enhancement of sensitivity to tumor necrosis factor alpha in non-small cell lung cancer cells with acquired resistance to gefitinib. Clin Cancer Res. 11(24 Pt 1): 8872-9). The combination of TNFα and chemotherapeutic agents has been shown to be an effect therapeutic strategy for many tumors by increasing tumor sensitivity to treatment. TNFα is also capable of activating T cells and dendritic cells to enhance host antitumor adaptive immune response.

The findings presented herein show that M0 and M2a macrophages can be stimulated to secrete TNFα and that alternatively activated M2a macrophages are the only cell type that upregulate MCP-1 upon cross-linking by IgE. Thus, IgE has the capacity specifically to stimulate this normally anti-inflammatory alternatively-activated macrophage subset to a more mature, more activated phenotype, which bears some characteristics normally associated with the classically-activated M1 macrophages. Such a finding suggests a role for IgE-mediated polarisation of alternatively-activated macrophages in the treatment of cancer by targeting macrophages, for example the use of macrophage-centred immunotherapy to re-educate macrophages into activatory phenotypes directly in tumours.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An immunoglobulin E (IgE) for use in repolarizing macrophages from a first phenotype to an anti-tumor phenotype in the treatment of cancer in a subject; wherein the first phenotype comprises a quiescent (M0) macrophage phenotype or an anti-inflammatory (M2a) macrophage phenotype; and the anti-tumor phenotype comprises a newly polarized macrophage phenotype characterized by expression of the following cytokines and chemokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10).

2. An immunoglobulin E (IgE) for use in the treatment of cancer in a subject; wherein macrophages associated with a tumor in the subject have a quiescent (M0) macrophage phenotype or an anti-inflammatory (M2a) macrophage phenotype; and the IgE treatment promotes repolarization of the macrophages associated with the tumor to a newly polarized macrophage phenotype characterized by expression of the following cytokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10).

3. An IgE for use according to claim 1 or claim 2, wherein the newly polarized macrophage phenotype is further characterized by increased expression of monocyte chemoattractant protein-1 (MCP-1), compared to an anti-inflammatory (M2a) macrophage phenotype.

4. An IgE for use according to any preceding claim, wherein the newly polarized macrophage phenotype is further characterized by increased expression of interleukin-12 (IL-12), interleukin-13 (IL-13), Chemokine (C-X-C motif) Ligand 9 (CXCL9) and/or Chemokine (C-X-C motif) Ligand 11 (CXCL11) compared to a quiescent (M0) macrophage phenotype or an anti-inflammatory (M2a) macrophage phenotype.

5. An IgE for use according to any preceding claim, wherein the IgE treatment further promotes increased expression of IFNγ and/or IL-12 by pro-inflammatory (M1) macrophages associated with a tumor in the subject.

6. An IgE for use according to any preceding claim, wherein macrophages associated with a tumor in the subject are distributed around a periphery of the tumor.

7. An IgE for use according to any preceding claim, wherein the newly polarized macrophage phenotype promotes further monocyte and/or macrophage recruitment into a tumor in the subject.

8. An IgE for use according to any preceding claim, wherein the cancer comprises skin cancer, breast cancer, head and neck squamous cell carcinoma, prostate cancer, ovarian cancer, colon cancer, glioma, stomach cancer, lung cancer or pancreatic cancer.

9. An IgE for use according to any preceding claim, wherein the IgE comprises an anti-folate receptor α (FRα) antibody, an anti-high molecular weight melanoma associated antigen (HMW-MAA) antibody, an anti-human epidermal growth factor receptor 2 (HER2) antibody or an anti-SF-25 antibody.

10. A method for treating cancer in a subject in need thereof, the method comprising administering a therapeutically effective amount of an immunoglobulin E (IgE) to the subject, wherein macrophages associated with a tumor in the subject have a quiescent (M0) macrophage phenotype or an anti-inflammatory (M2a) macrophage phenotype; and the IgE treatment promotes repolarization of the macrophages associated with the tumor to a newly polarized macrophage phenotype characterized by expression of the following cytokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10); wherein the newly polarized macrophage phenotype has enhanced anti-tumor activity compared to the quiescent (M0) macrophage phenotype or anti-inflammatory (M2a) macrophage phenotype; thereby treating cancer in the subject.

11. A method according to claim 10, wherein the method comprises a step of detecting one or more phenotypes of macrophages present in a tumor sample obtained from the subject; and administering the IgE to the subject if quiescent (M0) and/or inflammatory (M2a) macrophages are present in the sample at above a predetermined level.

12. A method according to claim 11, wherein the method comprises detecting expression of one or more of the following cytokines by macrophages present in the sample: TNFα; IFNγ, IL-1β, IL-6; RANTES, IL-10, MCP-1, IL-4, IL-13, MCP-1, CXCL9, IL-12 and/or CXCL11.

13. An immunoglobulin E (IgE) for use in repolarising macrophages associated with a tumor in a subject, wherein the repolarization results in modulation of cytokine expression in the tumor microenvironment and enhanced anti-tumor activity.

14. An IgE for use according to claim 13, wherein the repolarized macrophages express tumor necrosis factor alpha (TNFα).

15. An IgE for use according to claim 13 or claim 14, wherein the repolarized macrophages express interferon-gamma (IFNγ).

16. An IgE for use according to any of claims 13 to 15, wherein the repolarized macrophages express interleukin-1beta (IL-1β).

17. An IgE for use according to any of claims 13 to 16, wherein the repolarized macrophages express interleukin-6 (IL-6).

18. An IgE for use according to any of claims 13 to 17, wherein the repolarized macrophages express Regulated on Activation, Normal T cell Expressed and Secreted (RANTES or CCL5).

19. An IgE for use according to any of claims 13 to 18, wherein the repolarized macrophages express interleukin-10 (IL-10).

20. An IgE for use according to claims 13 to 19, wherein the repolarized macrophages comprise a newly polarized macrophage phenotype characterized by expression of the following cytokines and chemokines: tumor necrosis factor alpha (TNFα); interferon-gamma (IFNγ); interleukin-1beta (IL-1β); interleukin-6 (IL-6); Regulated on Activation, Normal T cell Expressed and Secreted (RANTES or CCL5); and interleukin-10 (IL-10).

21. An IgE for use according to any of claims 13 to 20, wherein the repolarized macrophages express monocyte chemoattractant protein-1 (MCP-1).
